SEQUENCE LISTING (4 sequences)

SEQ ID NO: 1 - protein, 451 aa, Artificial Sequence
  humanized antibody sequence
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 2 - protein, 214 aa, Artificial Sequence
  humanized antibody sequence
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 3 - protein, 451 aa, Artificial Sequence; identical to SEQ ID NO: 1
  humanized antibody sequence

SEQ ID NO: 4 - protein, 214 aa, Artificial Sequence; identical to SEQ ID NO: 2
  humanized antibody sequence

(19) United States
(12) Patent Application Publication
(10) Pub. No.: US 2013/0089496 A1
(43) Pub. Date: Apr. 11, 2013

(54) TUMOR TARGETED ANTIBODIES AND METHOD FOR USING THE SAME

(71) Applicant: Musc Foundation For Research Development, Charleston, SC (US)

(72) Inventor: Janardan P. PANDEY, Charleston, SC (US)

(73) Assignee: MUSC FOUNDATION FOR RESEARCH DEVELOPMENT, Charleston, SC (US)

(21) Appl. No.: 13/645,814

(22) Filed: Oct. 5, 2012

Related U.S. Application Data
(60) Provisional application No. 61/545,000, filed on Oct. 7, 2011.

(51) Int. Cl. C07K 16/40

(52) U.S. Cl. CPC C07K 16/40
USPC 424/1.49; 424/138.1; 424/133.1; 424/178.1

(57) ABSTRACT

Isolated and recombinant antibodies, such as EGFR-binding antibodies. Antibodies can comprising the sequence of an immunoglobulin heavy constant gamma-1 region encoded by a human G1M3 allele. For example, antibodies of the embodiments can comprise an immunoglobulin heavy constant gamma-1 region wherein position 214 is arginine; position 356 is glutamic acid; position 358 is methionine and/or position 431 is alanine. Methods for treating diseases, such as cancer, in a human subject with such antibodies are also provided.

This application claims the benefit of U.S. Provisional Patent Application No. 61/545,000, filed Oct. 7, 2011, the entirety of which is incorporated herein by reference.

The invention was made with government support under Grant Nos. W81XWH-08-1-0373 and W81XWH-09-1-0329 awarded by the Department of Defense. The government has certain rights in the invention.

### BACKGROUND OF THE INVENTION

1. Field of the Invention

The present invention relates generally to the field of molecular biology, and immunology. More particularly, it concerns molecule antibody design and antibody-based therapeutics.

2. Description of Related Art

Antibody-dependent cell-mediated cytotoxicity (ADCC), which links the specific humoral responses to the vigorous innate cytotoxic effector responses, is a major host defense mechanism against tumors. IgG antibody mediated ADCC is triggered upon binding of FcγR to the Fc of IgG molecules (Nimmerjahn and Ravetch, 2008). A range of modern cancer therapies have exploited this host defense by designing tumor targeted antibodies that can be used to mediate ADCC, even in the absence of a host immune response.

In the case of breast cancers one of the primary receptors targeted for therapy has been Her2/neu (ErbB2). At least 25% of breast cancers are ErbB2 positive, thus, targeting of this receptor has proven a fruitfull avenue for the development of therapeutics. Trastuzumab (Herceptin®), for example, is a humanized monoclonal ErbB2-binding antibody that has proven highly effective for targeted therapy of breast cancer. However, despite the effectiveness of ErbB2-targeted therapy, nearly 35% of breast cancer patients do not display an adequate response to the therapy. Consequently, methods are needed to improve the efficacy of targeted antibody therapies.

### SUMMARY OF THE INVENTION

In a first embodiment, there is provided an isolated or recombinant antibody comprising an immunoglobulin heavy constant gamma-1 region wherein position 214 is arginine; position 356 is glutamic acid; position 358 is methionine and/or position 431 is alanine. As used herein all numbering of amino acid position in an antibody refer to the standard nomenclature set forth, e.g., in Kabat et al., 1991, incorporated herein by reference. For example, the antibody can have an amino acid sequence corresponding to the human G1M3 allele coding sequence. In some aspects, an isolated or recombinant antibody of the embodiments is an antibody that binds to a tumor associated protein, such as epidermal growth factor receptor 1 (HER1) or HER2. In certain aspects, an isolated or recombinant antibody of the embodiments is a human antibody, a humanized antibody, a chimeric antibody, a single chain antibody or an antibody fragment, wherein the fragment comprises at the amino acids corresponding to positions 214, 356, 358 and/or 431 of the immunoglobulin heavy constant gamma-1 region.

In certain embodiments, there is provided an isolated or recombinant HER2-binding antibody wherein the antibody comprises an immunoglobulin heavy constant gamma-1 region (or a portion thereof) wherein position 214 is arginine; position 356 is glutamic acid; position 358 is methionine and/or position 431 is alanine. In some aspects, a HER2-binding antibody of the embodiments comprises 1, 2, 3, 4, 5, or all 6 of the CDRs of the Trastuzumab monoclonal antibody. For example, the HER2-binding antibody can comprises an amino acid sequence at least about 75%, 80%, 85%, 90%, 95%, 98% or 99% identical to the heavy chain and/or light chain amino acid sequence of the Trastuzumab monoclonal antibody (i.e., SEQ ID NOs: 1 and 2, respectively) wherein the amino acid at position(s) corresponding to immunoglobulin heavy constant gamma-1 region positions 214, 356, 358 and/or 431 are arginine, glutamic acid, methionine and/or alanine, respectively.

In some embodiments, there is provided an isolated or recombinant HER1-binding antibody wherein the antibody comprises an immunoglobulin heavy constant gamma-1 region (or a portion thereof) wherein position 214 is arginine; position 356 is glutamic acid; position 358 is methionine and/or position 431 is alanine. In some aspects, a HER1-binding antibody of the embodiments comprises 1, 2, 3, 4, 5, or all 6 of the CDRs of the Cetuximab monoclonal antibody. For example, the HER1-binding antibody can comprises an amino acid sequence at least about 75%, 80%, 85%, 90%, 95%, 98% or 99% identical to the heavy chain and/or light chain amino acid sequence of the Cetuximab monoclonal antibody (i.e., SEQ ID NOs: 3 and 4, respectively) wherein the amino acid(s) at positions corresponding to immunoglobulin heavy constant gamma-1 region positions 214, 356, 358 and/or 431 are arginine, glutamic acid, methionine and/or alanine, respectively.

In certain aspects an antibody according to the embodiments is coupled to a therapeutic, a reporter, or a targeting moiety. For example, the therapeutic can be a polynucleotide (e.g., an miRNA, a siRNA or an therapeutic gene), a peptide, a small molecule, a therapeutic radionuclide, a chemotherapeutic, a tumor suppressor, an apoptosis inducer, an enzyme, a second antibody, an siRNA, a hormone, a prodrug, or an immunostimulant. Examples of reporters include, but are not limited to, radionuclides, florophores, MRI contrast agents, enzymes, dyes or molecules detectable by positron emission tomography (PET).

In a further embodiment there is provided an isolated polynucleotide molecule encoding an antibody of the embodiments. In certain aspects the isolated polynucleotide further encodes a promoter, enhancer, polyadenylation sequence, intron, drug selection marker, origin of replication, a reporter or a purification tag. For example, the isolated polynucleotide can be comprised in a polynucleotide expression vector.

In some embodiments, the invention provides a pharmaceutical composition comprising an isolated or recombinant antibody of the embodiments in a pharmaceutically acceptable carrier.

In a further embodiment there is provided a method of treating an epidermal growth factor receptor (EGFR)-expressing cancer in a human, comprising administering to the human a therapeutically effective amount of an EGFR-binding antibody comprising an immunoglobulin heavy constant gamma-1 region wherein position 214 is arginine; position 356 is glutamic acid; position 358 is methionine and/or position 431 is alanine. For example, a cancer for treatment according to the embodiments can be a breast, bladder, brain, lung, liver, spleen, kidney, lymph node, small intestine, pancreas, blood cells, colon, stomach, breast, endometrium, prostate, testicle, ovary, skin, head and neck, esophagus, bone marrow or blood cancer. In certain aspects, a cancer for treatment according to the embodiments is an EGFR-expressing cancer. In certain embodiments, at least 1%, 5%, 10%, 15%, 20%, 25%, 30%, 35%, 40%, 45%, 50%, 55%, 65%, 70%, 75%, 80%, 85%, 90%, 95%, 96%, 97%, 98%, or 99% tumor cells in the cancer express EGFR (e.g., HER1 or HER2).

In some aspects, a method of the embodiments further comprises determining a FcγRIIIA gene allelic pattern in the human. For example, the allelic pattern can be determined by allele specific hybridization, primer specific extension, oligonucleotides ligation assay, restriction enzyme site analysis or single-stranded conformation polymorphism analysis. In some aspects, determining FcγRIIIA gene allelic pattern in the human comprises determining the amino acid encoded at position 158 of an Fc gamma receptor IIIA in the human. For example, the method can comprise determining whether one or two alleles of the FcγRIIIA gene encode a phenylalanine (F) or a valine (V) at position 158.

In a further embodiment, there is provided a method for treating a human subject having a cancer, wherein it was previously determined that one or two alleles of the FcγRIIIA gene of the subject encode a phenylalanine (F) or a valine (V) at position 158, the method comprising administering a therapeutically effective amount of an tumor antigen-binding antibody comprising an immunoglobulin heavy constant gamma-1 region wherein position 214 is arginine; position 356 is glutamic acid; position 358 is methionine and/or position 431 is alanine. For example, in certain aspects, a method of the embodiments comprises treating a patent that was previously determined to have at least one allele of the FcγRIIIA gene that encodes a phenylalanine (F) at position 158.

In yet still a further embodiment there is provided a method for treating a human subject having a cancer, wherein it was previously determined that the cancer expresses an EGFR, the method comprising administering a therapeutically effective amount of an EGFR-binding antibody of the embodiments. Methods for assessing the EGFR-expression status of a cancer have been described, for example in U.S. Patent Publn. No. 20110052570, incorporated herein by reference. In certain aspects, the EGFR-expressing cancer can be a cancer that expresses a mutant EGFR, such as a cancer expressing an EGFR having a L858R and/or T790M mutation.

Antibodies and pharmaceutical compositions according to the embodiments can be administered to a patent by a variety of routes including, without limitation, by intravenous administration, intracardiac administration, intradermal administration, intralesional administration, intrathecal administration, intracranial administration, intrapericardial administration, intraumbilical administration, intraocular administration, intraarterial administration, intraperitoneal administration, intraosseous administration, intrahemmorhage administration, intratrauma administration, intratumor administration, subcutaneous administration, intramuscular administration, intravitreous administration, direct injection into a normal tissue, or by direct injection into a tumor.

In further aspects, a method provided herein further comprises administering at least a second anticancer therapy. For example, the second anticancer therapy may be a surgical therapy, chemotherapy, radiation therapy, cryotherapy, hyperthermia treatment, phototherapy, radioablation therapy, hormonal therapy, immunotherapy, small molecule therapy, receptor kinase inhibitor therapy, anti-angiogenic therapy, cytokine therapy or a biological therapies such as monoclonal antibodies, siRNA, antisense oligonucleotides, ribozymes or gene therapy. Without limitation the biological therapy may be a gene therapy, such as tumor suppressor gene therapy, a cell death protein gene therapy, a cell cycle regulator gene therapy, a cytokine gene therapy, a toxin gene therapy, an immunogene therapy, a suicide gene therapy, a prodrug gene therapy, an anti-cellular proliferation gene therapy, an enzyme gene therapy, or an anti-angiogenic factor gene therapy. For example, in certain aspects, the second anticancer therapy can comprise administration of an EGFR inhibitor (e.g., a small molecule inhibitor).

It is contemplated that any method or composition described herein can be implemented with respect to any other method or composition described herein. Likewise, aspects of the present embodiments discussed in the context of a method for treating a subject are equally applicable to pharmaceutical compositions of the embodiments.

The use of the word “a” or “an” when used in conjunction with the term “comprising” in the claims and/or the specification may mean “one,” but it is also consistent with the meaning of “one or more,” “at least one,” and “one or more than one.”

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### DESCRIPTION OF ILLUSTRATIVE EMBODIMENTS

Antibodies that target specific cell types are currently used in a range of modern medical therapies, such as anticancer therapies. Despite the targeting specificity of such antibodies, many fail to exhibit sufficient effect (e.g., cell killing) on target cells. The inventors have now demonstrated that changes in antibody heavy chain constant regions, such as those resulting from allelic variation, impact the ability of the antibody to mediate effect on target cells. In particular, certain Fc coding sequences bind more effectively to Fc receptor molecules, including those expressed on natural killer (NK) cells, that mediate cell killing. Antibodies including these Fc sequences can therefore more effectively facilitate the killing of target cells in vivo.

Studies presented herein evaluated the influence of allotypically disparate IgG1 proteins on antibody-dependent cell-mediated cytotoxicity (ADCC). In particular, it was determined how effectively IgG-1 proteins with different Fc sequences compete with trastuzumab bound to target cells. Likewise, IgG-1 proteins with different Fc coding sequences were used to compete with cetuximab bound to the target cells. In both assays, cells were then contacted with NK cells expressing different variants of the FcγRIIIa Fc receptor. Thus, antibodies with Fc domains having a high affinity for the NK Fc receptor were able to outcompete the trastuzumab or cetuximab and thereby protect cells from killing (ADCC). Results of the studies showed that immunoglobulin comprising a heavy chain constant region encoded by the G1M3 allotype were most effective at competing for binding. Thus, antibodies comprising these high affinity sequences could be used for targeting cells and would mediate more effective cell killing. Importantly, the antibody variants identified in the studies were able to successfully compete for binding of FcγRIIIa receptors irrespective of the receptor genotype (i.e., FcγRIIIa-VV vs. FcγRIIIa-FF), thus antibodies including these coding sequences should be effective in humans having both of these allelic variants of the FcγRIIIa Fc receptor.

As demonstrated herein, the effectiveness of antibody therapeutics can be enhanced by providing an antibody comprising an Fc region with enhanced binding to Fc receptors. Examples, of such antibodies include an heavy constant region wherein position 214 is arginine; position 356 is glutamic acid; position 358 is methionine and/or position 431 is alanine (e.g., antibodies having a sequence corresponding to the human G1M3 allotype). Antibodies comprising these amino acid sequences are able to efficiently recruit immune cells, such as NK cells, to mediate effective killing of targeted cells. Moreover, because these antibodies bind to both the FcγRIIIa-VV and the FcγRIIIa-FF allotype, the antibodies offer significant efficacy in patients expressing either (or both) of these alleles. Thus, antibody therapeutics, such as trastuzumab or cetuximab, modified to comprise the amino acid coding sequence detailed here offer enhanced efficacy and efficacy in a broader range of patients.

### I. DEFINITIONS

The term “antigen” or “immunogen” as used herein is defined as a molecule that provokes an immune response when it is introduced into a subject or produced by a subject (tumor antigens arise by the cancer development itself). This immune response may involve either antibody production, or the activation of specific immunologically-competent cells, or both. An antigen can be derived from organisms, subunits of proteins/antigens, killed or inactivated whole cells or lysates, although in the present invention the antigen is a tumor antigen on the surface of a cancer cell. Commonly, an antigen is a molecule that causes the subject in which it is introduced to produce antibodies that specifically recognize the antigen. The part of the antigen with which the antibody interacts is termed an “epitope” or “antigenic determinant”. A skilled artisan realizes that any macromolecule, including virtually all proteins or peptides, can serve as antigens. Furthermore, antigens can be derived from recombinant or genomic DNA. A skilled artisan realizes that any DNA that contains nucleotide sequences or partial nucleotide sequences of a pathogenic genome or a gene or a fragment of a gene for a protein that elicits an immune response results in synthesis of an antigen.

The term “antigenic” and “immunogenic” as used herein describe a structure that is an antigen. These terms can be used interchangeably.

The term “antibody” as used herein refers to an immunoglobulin molecule, which is able to bind to a specific antigen. As used herein, an antibody is intended to refer broadly to any immunologic binding agent such as IgG, IgM, IgA, IgD and IgE. Antibodies can be intact immunoglobulins derived from natural sources or from recombinant sources and can be immunoactive portions of intact immunoglobulins. Antibodies are typically tetramers of immunoglobulin molecules. The antibodies in the present invention may exist in a variety of forms including, for example, polyclonal antibodies, monoclonal antibodies, and fragments thereof, as well as single chain antibodies and humanized antibodies (Harlow and Lane, 1988; Bird et al., 1988).

As used herein, the term “pharmaceutically or pharmacologically acceptable” refers to molecular entities and compositions that do not produce adverse, allergic, or other untoward reactions when administered to an animal or a human.

The term “pharmaceutically acceptable carrier” or “pharmaceutically acceptable excipient” or “physiologically acceptable carrier” as used herein includes any and all solvents, dispersion media, coatings, surfactants, antioxidants, preservatives (e.g., antibacterial agents, antifungal agents), isotonic agents, absorption delaying agents, salts, preservatives, drugs, drug stabilizers, gels, binders, excipients, disintegration agents, lubricants, dyes, such like materials and combinations thereof, as would be known to one of ordinary skill in the art (see, for example, Remington's Pharmaceutical Sciences, 18th Ed. Mack Printing Company, 1990, pp. 1289-1329, incorporated herein by reference). Except insofar as any conventional carrier is incompatible with the active ingredient, its use in the therapeutic or pharmaceutical compositions is contemplated. The carrier may not produce an adverse, allergic or other untoward reaction when administered to an animal, such as, for example, a human, as appropriate. These terms can be used interchangeably.

The term “subject” or “individual,” as used herein refers to animals, including mammals. More specifically, mammals include, but are not limited to rats, mice, rabbits, cats, dogs, monkeys and humans. These terms can be used interchangeably.

### II. ANTIBODIES FOR IMMUNOTHERAPY

The present invention provides antibodies to be used as immunotherapy for hyperproliferative diseases and disorders (e.g., cancer). The antibodies of the present invention are immunogically reactive with a tumor antigen, such as an EGFR. Thus, in one aspect, the invention is directed to a humanized monoclonal antibody immunoreactive with a tumor antigen. In certain aspects, antibodies of the embodiments comprise at least a portion of the constant region, such as region corresponding to immunoglobulin heavy constant gamma-1 region position 214, 356, 358 and 431. Antibodies of the embodiments can for example comprise an amino acid corresponding to position 214, 356, 358 and/or 431 of the constant gamma-1 region that is an arginine, glutamic acid, methionine and/or alanine, respectively. Thus, an antibody can comprise a constant domain comprising any of the sequences as indicated in Table 1 below.

TABLE 1
Immunoglobulin constant domain sequences.
IgG-1 constant region sequence

| IgG-1 constant | | | | |
|---|---|---|---|---|
| region sequence | 214—Arg | 356—Glu | 358—Met | 431—Ala |
| 214—Arg | | x | x | x |
| | | | x | x |
| | | | | x |
| | | | x | |
| | | x | | |
| | | x | x | |
| | | x | | x |
| 356—Glu | x | | x | x |
| | | | x | x |
| | | | | x |
| | | | x | |
| | x | | | |
| | x | | x | |
| | x | | | x |
| 358—Met | x | x | | x |
| | | x | | x |
| | | | | x |
| | | x | | |
| | x | | | |
| | x | x | | |
| | x | | | x |
| 431—Ala | x | x | x | |
| | | x | x | |
| | | | x | |
| | | x | | |
| | x | | | |
| | x | x | | |
| | x | | x | |

As used herein the term “humanized” is directed to antibodies or fragments immunospecific for a tumor antigen. Thus, the humanized antibodies or immunoreactive fragments of the invention are compatible with the human immune system. By “compatible with the human immune system” is meant that the antibodies or fragments of the invention do not elicit a substantial immune response when administered to humans as compared to unmodified forms of nonhuman antibodies containing the same complementarity-determining regions (CDRs). Eliciting an immune response is clearly undesirable as antibodies raised against therapeutically administered materials undermine the effectiveness of the administered materials and in addition may provoke unwanted side-effects due to stimulation of the immune system per se. While the antibodies and fragments of the invention may not, of course, be completely neutral with respect to an immune response in a specific individual, their effect on the immune system of an individual will be substantially less than that elicited by corresponding nonhuman antibodies in their unmodified forms.

Yet further, as used herein, the term “fully human antibody” or “fully humanized antibody” refers to antibodies or fragments immunospecific for human tumor antigen that have relatively no CDR or FR residues substituted from analogous sites in nonhuman species. Thus, the human variable domain is intact.

As discussed herein, minor variations in the amino acid sequences of antibodies or immunoglobulin molecules are contemplated as being encompassed by the humanized antibodies of the present invention, providing that the variations in the amino acid sequence maintain at least 75%, more preferably at least 80%, 90%, 95%, and most preferably 99% homology to the human variable domain. Specifically, in the present invention if the humanized antibody maintains at least 95% and most preferably 99% homology to the human variable domain, then the humanized antibody is considered to be fully humanized.

In particular, the variations that may be contemplated are conservative amino acid replacements. Conservative replacements are those that take place within a family of amino acids that are related in their side chains. Genetically encoded amino acids are generally divided into families: (1) acidic=aspartate, glutamate (glutamic acid); (2) basic=lysine, arginine, histidine; (3) non-polar=alanine, valine, leucine, isoleucine, proline, phenylalanine, methionine, tryptophan; and (4) uncharged polar=lysine, asparagine, glutamine, cysteine, serine, threonine, tyrosine. More particular families are: serine and threonine are aliphatic-hydroxy family; asparagine and glutamine are an amide-containing family; alanine, valine, leucine and isoleucine are an aliphatic family; and phenylalanine, tryptophan, and tyrosine are an aromatic family. For example, it is reasonable to expect that an isolated replacement of a leucine with an isoleucine or valine, an aspartate with a glutamate, a threonine with a serine, or a similar replacement of an amino acid with a structurally related amino acid will not have a major effect on the binding or properties of the resulting molecule, especially if the replacement does not involve an amino acid within a framework site. Whether an amino acid change results in a functional peptide can readily be determined by assaying the specific activity of the polypeptide derivative. Fragments or analogs of antibodies or immunoglobulin molecules can be readily prepared by those of ordinary skill in the art. Particular amino- and carboxy-termini of fragments or analogs occur near boundaries of functional domains. Structural and functional domains can be identified by comparison of the nucleotide and/or amino acid sequence data to public or proprietary sequence databases. Preferably, computerized comparison methods are used to identify sequence motifs or predicted protein conformation domains that occur in other proteins of known structure and/or function. Methods to identify protein sequences that fold into a known three-dimensional structure are known (Bowie et al., 1991).

Particular amino acid substitutions are those such as follows: (1) reduce susceptibility to proteolysis, (2) reduce susceptibility to oxidation, (3) alter binding affinity for forming protein complexes, (4) alter binding affinities, and (4) confer or modify other physiocochemical or functional properties of such analogs. Analogs can include various mutations of a sequence other than the naturally-occurring peptide sequence. For example, single or multiple amino acid substitutions (preferably conservative amino acid substitutions) may be made in the naturally-occurring sequence (preferably in the portion of the polypeptide outside the domain(s) forming intermolecular contacts. A conservative amino acid substitution should not substantially change the structural characteristics of the parent sequence (e.g., a replacement amino acid should not tend to break a helix that occurs in the parent sequence, or disrupt other types of secondary structure that characterizes the parent sequence).

### III. ANTIBODY PREPARATION

In certain aspects, the antibodies of the present invention may be produced using standard procedures that are well known and used in the art. For example, an antigen-binding antibody can be produced as detailed below and then the antieg-specifc CDRs can be engineered into a human or humanized antibody according to the embodiments.

A. Polyclonal Antibodies

Polyclonal antibodies that react immunologically to a tumor antigen generally are raised in animals by multiple subcutaneous (sc) or intraperitoneal (ip) injections of the tumor antigen and an adjuvant.

Animals are immunized against the immunogenic composition or derivatives. Animals are boosted until the titer plateaus. The animals are usually bled through an ear vein or alternatively by cardiac puncture. The removed blood is allowed to coagulate and then centrifuged to separate serum components from whole cells and blood clots. The serum may be used as is for various applications or else the desired antibody fraction may be purified by well-known methods, such as affinity chromatography using another antibody, a peptide bound to a solid matrix, or by using, e.g., protein A or protein G chromatography.

B. Monoclonal Antibodies

The methods for generating monoclonal antibodies (MAbs) generally begin along the same lines as those for preparing polyclonal antibodies. Rodents such as mice and rats are particular animals, however, the use of rabbit, sheep, goat, monkey cells also is possible. The use of rats may provide certain advantages (Goding, 1986), but mice are used, with the BALB/c mouse being most routinely used and generally gives a higher percentage of stable fusions.

Monoclonal antibodies are obtained from a population of substantially homogeneous antibodies, i.e., the individual antibodies comprising the population are identical except for possible naturally-occurring mutations that may be present in minor amounts. Thus, the modifier “monoclonal” indicates the character of the antibody as not being a mixture of discrete antibodies. Methods for generating monoclonal antibodies are described elsewhere herein.

C. Humanized Antibodies

Methods for humanizing non-human antibodies are well known in the art. Generally, a humanized antibody has one or more amino acid residues introduced into it from a source, which is non-human. These non-human amino acid residues are often referred to as “import” residues, which are typically taken from an “import” variable domain. Humanization can be essentially performed following the method of Winter and co-workers (Jones et al., 1986; Riechmann et al., 1988; Verhoeyen et al., 1988), by substituting rodent CDRs or CDR sequences for the corresponding sequences of a human antibody. Accordingly, such “humanized” antibodies are chimeric antibodies wherein substantially less than an intact human variable domain has been substituted by the corresponding sequence from a non-human species. In practice, humanized antibodies are typically human antibodies in which some CDR residues and possibly some FR residues are substituted by residues from analogous sites in rodent antibodies.

It may be beneficial that antibodies be humanized with retention of high affinity for the antigen and other favorable biological properties. To achieve this goal, according to a particular method, humanized antibodies are prepared by a process of analysis of the parental sequences and various conceptual humanized products using three-dimensional models of the parental and humanized sequences. Three dimensional immunoglobulin models are commonly available and are familiar to those skilled in the art. Computer programs are available which illustrate and display probable three-dimensional conformational structures of selected candidate immunoglobulin sequences. Inspection of these displays permits analysis of the likely role of the residues in the functioning of the candidate immunoglobulin sequence, i.e. the analysis of residues that influence the ability of the candidate immunoglobulin to bind its antigen.

D. Human Antibodies

Human monoclonal antibodies can be made by the hybridoma method. Human myeloma and mouse-human heteromyeloma cell lines for the production of human monoclonal antibodies have been described (Kozbor, 1984; U.S. Pat. No. 6,150,584, which is incorporated herein by reference).

It is now possible to produce transgenic animals (e.g., mice) that are capable, upon immunization, of producing a repertoire of human antibodies in the absence of endogenous immunoglobulin production. For example, it has been described that the homozygous deletion of the antibody heavy chain joining region (JH) gene in chimeric and germ-line mutant mice results in complete inhibition of endogenous antibody production. Transfer of the human germ-line immunoglobulin gene array in such germ-line mutant mice will result in the production of human antibodies upon antigen challenge (Jakobovits et al., 1993).

Alternatively, the phage display technology (McCafferty et al., 1990) can be used to produce human antibodies and antibody fragments in vitro, from immunoglobulin variable (V) domain gene repertoires from unimmunized donors. According to this technique, antibody V domain genes are cloned in-frame into either a major or minor coat protein gene of a filamentous bacteriophage, such as M13 or fd, and displayed as functional antibody fragments on the surface of the phage particle.

### IV. IMMUNOTHERAPY TREATMENTS

A. Treatment of Hyperproliferative Diseases

In certain embodiments, a hyperproliferative disease may be treated by administering to a subject an effective amount of antibodies that react immunologically with a tumor antigen. The subject is preferably a mammal and more preferably a human.

In the present invention, a hyperproliferative disease is further defined as cancer. In still further embodiments, the cancer is melanoma, non-small cell lung, small-cell lung, lung, leukemia, hepatocarcinoma, retinoblastoma, astrocytoma, glioblastoma, gum, tongue, neuroblastoma, head, neck, breast, pancreatic, prostate, renal, bone, testicular, ovarian, mesothelioma, cervical, gastrointestinal, lymphoma, brain, colon, sarcoma or bladder.

The cancer may include a tumor comprised of tumor cells. For example, tumor cells may include, but are not limited to melanoma cell, a bladder cancer cell, a breast cancer cell, a lung cancer cell, a colon cancer cell, a prostate cancer cell, a liver cancer cell, a pancreatic cancer cell, a stomach cancer cell, a testicular cancer cell, a brain cancer cell, an ovarian cancer cell, a lymphatic cancer cell, a skin cancer cell, a brain cancer cell, a bone cancer cell, or a soft tissue cancer cell.

In a particular embodiment of the present invention, antibodies that react immunologically to a tumor antigen are administered in an effective amount to decrease, reduce, inhibit or abrogate the growth of cancer, including of a solid tumor. Examples of solid tumors that can be treated according to the invention include sarcomas and carcinomas such as, but not limited to: fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, cervical cancer, testicular tumor, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, melanoma, neuroblastoma, and retinoblastoma.

Yet further, hyperproliferative diseases that are most likely to be treated in the present invention are those that metastasize. It is understood by those in the art that metastasis is the spread of cells from a primary tumor to a noncontiguous site, usually via the bloodstream or lymphatics, which results in the establishment of a secondary tumor growth. Examples of hyperproliferative diseases contemplated for treatment include, but are not limited to melanoma, bladder, non-small cell lung, small cell lung, lung, hepatocarcinoma, retinoblastoma, astrocytoma, glioblastoma, neuroblastoma, head, neck, breast, pancreatic, gum, tongue, prostate, renal, bone, testicular, ovarian, mesothelioma, cervical, gastrointestinal lymphoma, brain, or colon cancer and any other hyperproliferative diseases that may be treated by administering an antibody that reacts immunologically with a tumor antigen.

B. Treatment Regimens

Treatment regimens may vary as well, and often depend on tumor type, tumor location, disease progression, and health and age of the patient. Obviously, certain types of tumor will require more aggressive treatment, while at the same time, certain patients cannot tolerate more taxing protocols. The clinician will be best suited to make such decisions based on the known efficacy and toxicity (if any) of the therapeutic formulations.

In certain aspects, patients to be treated will have adequate bone marrow function (defined as a peripheral absolute granulocyte count of >2,000/mm³ and a platelet count of 100,000/mm³), adequate liver function (bilirubin <1.5 mg/dl) and adequate renal function (creatinine <1.5 mg/dl).

As used herein the term “effective amount” is defined as an amount of the agent that will decrease, reduce, inhibit or otherwise abrogate the growth of a cancer cell, induce apoptosis, inhibit angiogenesis of a tumor cell, inhibit metastasis, or induce cytotoxicity in cells. Thus, an effective amount is an amount sufficient to detectably and repeatedly ameliorate, reduce, minimize or limit the extent of the disease or its symptoms. More rigorous definitions may apply, including elimination, eradication or cure of disease.

To kill cells, inhibit cell growth, inhibit metastasis, decrease tumor or tissue size and otherwise reverse or reduce the malignant phenotype of tumor cells, using the methods and compositions of the present invention, one would generally contact a tumor cell with an antibody that reacts immunologically thereto. The routes of administration will vary, naturally, with the location and nature of the lesion, and include, e.g., intradermal, transdermal, parenteral, intravenous, intramuscular, intranasal, subcutaneous, percutaneous, intratracheal, intraperitoneal, intratumoral, perfusion, lavage, direct injection, and oral administration and formulation.

In the case of surgical intervention, the present invention may be used preoperatively, to render an inoperable tumor subject to resection. Alternatively, the present invention may be used at the time of surgery, and/or thereafter, to treat residual or metastatic disease. For example, a resected tumor bed may be injected or perfused with a formulation comprising an antibody that reacts immunologically with a tumor antigen. The perfusion may be continued post-resection, for example, by leaving a catheter implanted at the site of the surgery. Periodic post-surgical treatment also is envisioned.

Continuous administration also may be applied where appropriate, for example, where a tumor is excised and the tumor bed is treated to eliminate residual, microscopic disease. Delivery via syringe or catherization is in particular contemplated. Such continuous perfusion may take place for a period from about 1-2 hours, to about 2-6 hours, to about 6-12 hours, to about 12-24 hours, to about 1-2 days, to about 1-2 wk or longer following the initiation of treatment. Generally, the dose of the therapeutic composition via continuous perfusion will be equivalent to that given by a single or multiple injections, adjusted over a period of time during which the perfusion occurs. It is further contemplated that limb perfusion may be used to administer therapeutic compositions of the present invention, particularly in the treatment of melanomas and sarcomas.

In certain embodiments, the tumor being treated may not, at least initially, be resectable. Treatments with therapeutic antibodies may increase the resectability of the tumor due to shrinkage at the margins or by elimination of certain particularly invasive portions. Following treatments, resection may be possible. Additional treatments subsequent to resection will serve to eliminate microscopic residual disease at the tumor site.

A typical course of treatment, for a primary tumor or a post-excision tumor bed, will involve multiple doses. Typical primary tumor treatment involves a 6 dose application over a two-week period. The two-week regimen may be repeated one, two, three, four, five, six or more times. During a course of treatment, the need to complete the planned dosings may be re-evaluated.

The treatments may include various “unit doses.” Unit dose is defined as containing a predetermined-quantity of the therapeutic composition. The quantity to be administered, and the particular route and formulation, are within the skill of those in the clinical arts. A unit dose need not be administered as a single injection but may comprise continuous infusion over a set period of time.

C. Treatment Regimen for Breast Cancer

It is envisioned that breast cancer, as only an exemplary cancer for treatment with the present invention, may be treated by employing the antibody treatment of the present invention. For example, antibodies that react immunologically to a tumor antigen may be employed at a starting dose of 1-3 mg/kg. Dosing may be every 3 weeks for 4 cycles (total=12 weeks), at which time response may also be determined. If no dose-limiting toxicity is observed after 2 cycles, then the next dosing level may be initiated according to standard dose-escalation algorithms (i.e., 3 mg/kg, 6 mg/kg, 9 mg/kg, 13.5 mg/kg, etc.).

In addition to toxicity and response data, tissue and serum samples are collected pre-therapy and post-therapy (after 2 and 4 cycles) to provide the basis for studies on intermediate biomarkers involved in angiogenesis and invasion and to evaluate whether these markers can predict response to treatment. To assess for alterations in blood flow, in situ, blood flow patterns are assessed in real time using 3-D re-constructions of high resolution cutaneous Doppler ultrasound examinations of accessible tumors pre-therapy and after 2 to 4 cycles.

### V. COMBINATION TREATMENTS

In some embodiments of the invention, it may be desirable to combine compositions for administration to the subject, particularly combining antibodies that react immunologically with a tumor antigen with other agents effective in the treatment of hyperproliferative disease, such as anti-cancer agents, or with surgery. An “anti-cancer” agent is capable of negatively affecting cancer in a subject, for example, by killing cancer cells, inducing apoptosis in cancer cells, reducing the growth rate of cancer cells, reducing the incidence or number of metastases, reducing tumor size, inhibiting tumor growth, reducing the blood supply to a tumor or cancer cells, promoting an immune response against cancer cells or a tumor, preventing or inhibiting the progression of cancer, or increasing the lifespan of a subject with cancer. Anti-cancer agents include biological agents (biotherapy), chemotherapy agents, and radiotherapy agents. More generally, these other compositions would be provided in a combined amount effective to kill or inhibit proliferation of the cell. This process may involve contacting the cells with the antibodies of the present invention and the agent(s) or multiple factor(s) at the same time. This may be achieved by contacting the cell with a single composition or pharmacological formulation that includes both agents, or by contacting the cell with two distinct compositions or formulations, at the same time, wherein one composition includes the antibodies and the other includes the second agent(s).

The antibodies of the present invention may precede or follow the other anti-cancer agent treatment by intervals ranging from minutes to weeks. In embodiments where the other anti-cancer agent and antibodies are applied separately to the cell, one would generally ensure that a significant period of time did not expire between the time of each delivery, such that the agent and antibodies would still be able to exert an advantageously combined effect on the cell. In such instances, it is contemplated that one may contact the cell with both modalities within about 12-24 h of each other and, more preferably, within about 6-12 h of each other. In some situations, it may be desirable to extend the time period for treatment significantly, however, where several d (2, 3, 4, 5, 6 or 7) to several wk (1, 2, 3, 4, 5, 6, 7 or 8) lapse between the respective administrations.

Various combinations may be employed, an antibody according to the embodiments is “A” and the secondary agent, such as radio-, chemotherapy or an additional tumor-targted antibody is “B”:

A/B/A B/A/B B/B/A A/A/B A/B/B B/A/A A/B/B/B B/A/B/B
B/B/B/A B/B/A/B A/A/B/B A/B/A/B A/B/B/A B/B/A/A
B/A/B/A B/A/A/B A/A/A/B B/A/A/A A/B/A/A A/A/B/A

Administration of the immunotherapy of the present invention to a patient will follow general protocols for the administration of chemotherapeutics. It is expected that the treatment cycles would be repeated as necessary. It also is contemplated that various standard therapies, as well as surgical intervention, may be applied in combination with the described hyperproliferative cell therapy.

A. Chemotherapy

Cancer therapies also include a variety of chemical based treatments. Some examples of chemotherapeutic agents include antibiotic chemotherapeutics such as Doxorubicin, Daunorubicin, Adriamycin, Mitomycin (also known as mutamycin and/or mitomycin-C), Actinomycin D (Dactinomycin), Bleomycin, Plicomycin, plant alkaloids such as Taxol, Vincristine, Vinblastine, miscellaneous agents such as Cisplatin (CDDP), etoposide (VP16), Tumor Necrosis Factor, and alkylating agents such as, Carmustine, Melphalan (also known as alkeran, L-phenylalanine mustard, phenylalanine mustard, L-PAM, or L-sarcolysin, is a phenylalanine derivative of nitrogen mustard), Cyclophosphamide, Chlorambucil, Busulfan (also known as myleran), and Lomustine.

Some examples of other agents include, but are not limited to, Carboplatin, Procarbazine, Mechlorethamine, Camptothecin, Ifosfamide, Nitrosurea, Etoposide (VP16), Tamoxifen, Raloxifene, Toremifene, Idoxifene, Droloxifene, TAT-59, Zindoxifene, Trioxifene, ICI 182,780, EM-800, Estrogen Receptor Binding Agents, Gemcitabien, Navelbine, Farnesyl-protein transferase inhibitors, Transplatinum, 5-Fluorouracil, hydrogen peroxide, and Methotrexate, Temazolomide (an aqueous form of DTIC), Mylotarg, Dolastatin-10, Bryostatin, or any analog or derivative variant of the foregoing.

B. Radiotherapeutic Agents

Radiotherapeutic agents and factors include radiation and waves that induce DNA damage for example, γ-irradiation, X-rays, UV-irradiation, microwaves, electronic emissions, radioisotopes, and the like. Therapy may be achieved by irradiating the localized tumor site with the above described forms of radiations. It is most likely that all of these factors effect a broad range of damage DNA, on the precursors of DNA, the replication and repair of DNA, and the assembly and maintenance of chromosomes.

Dosage ranges for X-rays range from daily doses of 50 to 200 roentgens for prolonged periods of time (3 to 4 weeks), to single doses of 2000 to 6000 roentgens. Dosage ranges for radioisotopes vary widely, and depend on the half-life of the isotope, the strength and type of radiation emitted, and the uptake by the neoplastic cells.

C. Surgery

Approximately 60% of persons with cancer will undergo surgery of some type, which includes preventative, diagnostic or staging, curative and palliative surgery. Curative surgery is a cancer treatment that may be used in conjunction with other therapies, such as the treatment of the present invention, chemotherapy, radiotherapy, hormonal therapy, gene therapy, immunotherapy and/or alternative therapies.

Curative surgery includes resection in which all or part of cancerous tissue is physically removed, excised, and/or destroyed. Tumor resection refers to physical removal of at least part of a tumor. In addition to tumor resection, treatment by surgery includes laser surgery, cryosurgery, electrosurgery, and miscopically controlled surgery (Mohs' surgery). It is further contemplated that the present invention may be used in conjunction with removal of superficial cancers, precancers, or incidental amounts of normal tissue.

Upon excision of part of all of cancerous cells, tissue, or tumor, a cavity may be formed in the body. Treatment may be accomplished by perfusion, direct injection or local application of the area with an additional anti-cancer therapy. Such treatment may be repeated, for example, every 1, 2, 3, 4, 5, 6, or 7 days, or every 1, 2, 3, 4, and 5 weeks or every 1, 2, 3, 4, 5, 6, 7, 8, 9, 10, 11, or 12 months. These treatments may be of varying dosages as well.

D. Gene Therapy

In yet another embodiment, gene therapy in conjunction with the combination therapy using the antibody compounds described in the invention are contemplated. A variety of proteins are encompassed within the invention, some of which are described below. Various genes that may be targeted for gene therapy of some form in combination with the present invention are known in the art, including p53, BRCA1, and/or BRCA2, for example.

### VI. IMMUNOLOGICAL REAGENTS

In certain aspects of the invention, one or more antibodies of the embodiments are employed for either therapeutic, prognostic, and/or diagnostic embodiments. Antibodies include any type of antibody, and specifically refer to antibodies that react immunologically with a tumor antigen, such as an EGFR (e.g., HER1 or HER2). In particular, these antibodies may be used in various diagnostic or therapeutic applications, described herein below.

As used herein, the term “antibody” is intended to refer broadly to any immunologic binding agent such as IgG, IgM, IgA, IgD and IgE. The term “antibody” is used to refer to any antibody-like molecule that has an antigen binding region, and includes antibody fragments such as Fab′, Fab, F(ab′)2, single domain antibodies (DABs), Fv, scFv (single chain Fv), and the like. The techniques for preparing and using various antibody-based constructs and fragments are well known in the art. Means for preparing and characterizing antibodies are also well known in the art (see, e.g., Harlow and Lane, 1988; incorporated herein by reference).

Monoclonal antibodies (MAbs) are recognized to have certain advantages, e.g., reproducibility and large-scale production. The invention thus provides monoclonal antibodies of the human, murine, monkey, rat, hamster, rabbit and even chicken origin. Due to the ease of preparation and ready availability of reagents, murine monoclonal antibodies will often be utilized.

However, “humanized” antibodies are also contemplated, as are chimeric antibodies from mouse, rat, or other species, bearing human constant and/or variable region domains, bispecific antibodies, recombinant and engineered antibodies and fragments thereof. Methods for the development of antibodies that are “custom-tailored” to the patient's dental disease are likewise known and such custom-tailored antibodies are also contemplated.

The methods for generating monoclonal antibodies (MAbs) generally begin along the same lines as those for preparing polyclonal antibodies. Briefly, a polyclonal antibody is prepared by immunizing an animal with a LEE or CEE composition in accordance with the present invention and collecting antisera from that immunized animal.

A wide range of animal species can be used for the production of antisera. Typically the animal used for production of antisera is a rabbit, a mouse, a rat, a hamster, a guinea pig or a goat. The choice of animal may be decided upon the ease of manipulation, costs or the desired amount of sera, as would be known to one of skill in the art.

As is also well known in the art, the immunogenicity of a particular immunogen composition can be enhanced by the use of non-specific stimulators of the immune response, known as adjuvants. Suitable adjuvants include all acceptable immunostimulatory compounds, such as cytokines, chemokines, cofactors, toxins, plasmodia, synthetic compositions or LEEs or CEEs encoding such adjuvants.

Adjuvants that may be used include IL-1, IL-2, IL-4, IL-7, IL-12, γ-interferon, GM-CSF, BCG, aluminum hydroxide, MDP compounds, such as thur-MDP and nor-MDP, CGP (MTP-PE), lipid A, and monophosphoryl lipid A (MPL). RIBI, which contains three components extracted from bacteria, MPL, trehalose dimycolate (TDM) and cell wall skeleton (CWS) in a 2% squalene/Tween 80 emulsion is also contemplated. MHC antigens may even be used. Exemplary adjuvants include complete Freund's adjuvant (a non-specific stimulator of the immune response containing killed Mycobacterium tuberculosis), incomplete Freund's adjuvants and aluminum hydroxide adjuvant.

In addition to adjuvants, it may be desirable to coadminister biologic response modifiers (BRM), which have been shown to upregulate T cell immunity or down-regulate suppressor cell activity. Such BRMs include, but are not limited to, Cimetidine (CIM; 1200 mg/d) (Smith/Kline, Pa.); low-dose Cyclophosphamide (CYP; 300 mg/m2) (Johnson/Mead, N.J.), cytokines such as γ-interferon, IL-2, or IL-12 or genes encoding proteins involved in immune helper functions, such as B-7.

The amount of immunogen composition used in the production of polyclonal antibodies varies upon the nature of the immunogen as well as the animal used for immunization. A variety of routes can be used to administer the immunogen including but not limited to subcutaneous, intramuscular, intradermal, intraepidermal, intravenous and intraperitoneal. The production of polyclonal antibodies may be monitored by sampling blood of the immunized animal at various points following immunization.

A second, booster dose (e.g., provided in an injection), may also be given. The process of boosting and titering is repeated until a suitable titer is achieved. When a desired level of immunogenicity is obtained, the immunized animal can be bled and the serum isolated and stored, and/or the animal can be used to generate MAbs.

For production of rabbit polyclonal antibodies, the animal can be bled through an ear vein or alternatively by cardiac puncture. The removed blood is allowed to coagulate and then centrifuged to separate serum components from whole cells and blood clots. The serum may be used as is for various applications or else the desired antibody fraction may be purified by well-known methods, such as affinity chromatography using another antibody, a peptide bound to a solid matrix, or by using, e.g., protein A or protein G chromatography.

MAbs may be readily prepared through use of well-known techniques, such as those exemplified in U.S. Pat. No. 4,196,265, incorporated herein by reference. Typically, this technique involves immunizing a suitable animal with a selected immunogen composition, e.g., a purified or partially purified protein, polypeptide, peptide or domain, be it a wild-type or mutant composition. The immunizing composition is administered in a manner effective to stimulate antibody producing cells.

The methods for generating monoclonal antibodies (MAbs) generally begin along the same lines as those for preparing polyclonal antibodies. Rodents such as mice and rats are often used, however, the use of rabbit, sheep or frog cells is also possible.

The animals are injected with antigen, generally as described above. The antigen may be mixed with adjuvant, such as Freund's complete or incomplete adjuvant. Booster administrations with the same antigen or DNA encoding the antigen would occur at approximately two-week intervals.

Following immunization, somatic cells with the potential for producing antibodies, specifically B lymphocytes (B cells), are selected for use in the MAb generating protocol. These cells may be obtained from biopsied spleens, tonsils or lymph nodes, or from a peripheral blood sample. Spleen cells and peripheral blood cells are preferred, the former because they are a rich source of antibody-producing cells that are in the dividing plasmablast stage, and the latter because peripheral blood is easily accessible.

Often, a panel of animals will have been immunized and the spleen of an animal with the highest antibody titer will be removed and the spleen lymphocytes obtained by homogenizing the spleen with a syringe. Typically, a spleen from an immunized mouse contains approximately 5×10⁷ to 2×10⁸ lymphocytes.

The antibody producing B lymphocytes from the immunized animal are then fused with cells of an immortal myeloma cell, generally one of the same species as the animal that was immunized. Myeloma cell lines suited for use in hybridoma producing fusion procedures preferably are non antibody producing, have high fusion efficiency, and enzyme deficiencies that render then incapable of growing in certain selective media which support the growth of only the desired fused cells (hybridomas).

Any one of a number of myeloma cells may be used, as are known to those of skill in the art (Goding, pp. 65-66, 1986; Campbell, pp. 75-83, 1984). For example, where the immunized animal is a mouse, one may use P3×63/Ag8, X63 Ag8.653, NS1/1.Ag 4 1, Sp210 Ag14, FO, NSO/U, MPC 11, MPC11×45 GTG 1.7 and S194/5XX0 Bul; for rats, one may use R210.RCY3, Y3 Ag 1.2.3, IR983F and 4B210; and U 266, GM1500 GRG2, LICR LON HMy2 and UC729 6 are all useful in connection with human cell fusions.

One particular murine myeloma cell is the NS-1 myeloma cell line (also termed P3-NS-1-Ag4-1), which is readily available from the NIGMS Human Genetic Mutant Cell Repository by requesting cell line repository number GM3573. Another mouse myeloma cell line that may be used is the 8 azaguanine resistant mouse murine myeloma SP2/0 non producer cell line.

Methods for generating hybrids of antibody producing spleen or lymph node cells and myeloma cells usually comprise mixing somatic cells with myeloma cells in a 2:1 proportion, though the proportion may vary from about 20:1 to about 1:1, respectively, in the presence of an agent or agents (chemical or electrical) that promote the fusion of cell membranes. Fusion methods using Sendai virus have been described by Kohler and Milstein (1975; 1976), and those using polyethylene glycol (PEG), such as 37% (v/v) PEG, by Gefter et al., (1977). The use of electrically induced fusion methods is also appropriate (Goding pp. 71-74, 1986).

Fusion procedures usually produce viable hybrids at low frequencies, about 1×10⁻⁶ to 1×10⁻⁸. However, this does not pose a problem, as the viable, fused hybrids are differentiated from the parental, unfused cells (particularly the unfused myeloma cells that would normally continue to divide indefinitely) by culturing in a selective medium. The selective medium is generally one that contains an agent that blocks the de novo synthesis of nucleotides in the tissue culture media. Exemplary agents are aminopterin, methotrexate, and azaserine. Aminopterin and methotrexate block de novo synthesis of both purines and pyrimidines, whereas azaserine blocks only purine synthesis. Where aminopterin or methotrexate is used, the media is supplemented with hypoxanthine and thymidine as a source of nucleotides (HAT medium). Where azaserine is used, the media is supplemented with hypoxanthine.

The favored selection medium is HAT. Only cells capable of operating nucleotide salvage pathways are able to survive in HAT medium. The myeloma cells are defective in key enzymes of the salvage pathway, e.g., hypoxanthine phosphoribosyl transferase (HPRT), and they cannot survive. The B cells can operate this pathway, but they have a limited life span in culture and generally die within about two weeks. Therefore, the only cells that can survive in the selective media are those hybrids formed from myeloma and B cells.

This culturing provides a population of hybridomas from which specific hybridomas are selected. Typically, selection of hybridomas is performed by culturing the cells by single-clone dilution in microtiter plates, followed by testing the individual clonal supernatants (after about two to three weeks) for the desired reactivity. The assay should be sensitive, simple and rapid, such as radioimmunoassays, enzyme immunoassays, cytotoxicity assays, plaque assays, dot immunobinding assays, and the like.

The selected hybridomas would then be serially diluted and cloned into individual antibody producing cell lines, which clones can then be propagated indefinitely to provide MAbs. The cell lines may be exploited for MAb production in two basic ways. First, a sample of the hybridoma can be injected (often into the peritoneal cavity) into a histocompatible animal of the type that was used to provide the somatic and myeloma cells for the original fusion (e.g., a syngeneic mouse). Optionally, the animals are primed with a hydrocarbon, especially oils such as pristane (tetramethylpentadecane) prior to injection. The injected animal develops tumors secreting the specific monoclonal antibody produced by the fused cell hybrid. The body fluids of the animal, such as serum or ascites fluid, can then be tapped to provide MAbs in high concentration. Second, the individual cell lines could be cultured in vitro, where the MAbs are naturally secreted into the culture medium from which they can be readily obtained in high concentrations.

MAbs produced by either means may be further purified, if desired, using filtration, centrifugation and various chromatographic methods such as HPLC or affinity chromatography. Fragments of the monoclonal antibodies of the invention can be obtained from the monoclonal antibodies so produced by methods which include digestion with enzymes, such as pepsin or papain, and/or by cleavage of disulfide bonds by chemical reduction. Alternatively, monoclonal antibody fragments encompassed by the present invention can be synthesized using an automated peptide synthesizer.

It is also contemplated that a molecular cloning approach may be used to generate monoclonals. In one embodiment, combinatorial immunoglobulin phagemid libraries are prepared from RNA isolated from the spleen of the immunized animal, and phagemids expressing appropriate antibodies are selected by panning using cells expressing the antigen and control cells. The advantages of this approach over conventional hybridoma techniques are that approximately 104 times as many antibodies can be produced and screened in a single round, and that new specificities are generated by H and L chain combination which further increases the chance of finding appropriate antibodies. In another example, LEEs or CEEs can be used to produce antigens in vitro with a cell free system. These can be used as targets for scanning single chain antibody libraries. This would enable many different antibodies to be identified very quickly without the use of animals.

Alternatively, monoclonal antibody fragments encompassed by the present invention can be synthesized using an automated peptide synthesizer, or by expression of full-length gene or of gene fragments in E. coli.

A. Antibody Conjugates

The present invention further provides antibodies to ORF transcribed messages and translated proteins, polypeptides and peptides, generally of the monoclonal type, that are linked to at least one agent to form an antibody conjugate. In order to increase the efficacy of antibody molecules as diagnostic or therapeutic agents, it is conventional to link or covalently bind or complex at least one desired molecule or moiety. Such a molecule or moiety may be, but is not limited to, at least one effector or reporter molecule. Effector molecules comprise molecules having a desired activity, e.g., cytotoxic activity. Non-limiting examples of effector molecules which have been attached to antibodies include toxins, anti-tumor agents, therapeutic enzymes, radio-labeled nucleotides, antiviral agents, chelating agents, cytokines, growth factors, and oligo- or poly-nucleotides. By contrast, a reporter molecule is defined as any moiety which may be detected using an assay. Non-limiting examples of reporter molecules which have been conjugated to antibodies include enzymes, radiolabels, haptens, fluorescent labels, phosphorescent molecules, chemiluminescent molecules, chromophores, luminescent molecules, photoaffinity molecules, colored particles or ligands, such as biotin.

Any antibody of sufficient selectivity, specificity or affinity may be employed as the basis for an antibody conjugate. Such properties may be evaluated using conventional immunological screening methodology known to those of skill in the art. Sites for binding to biological active molecules in the antibody molecule, in addition to the canonical antigen binding sites, include sites that reside in the variable domain that can bind pathogens, B-cell superantigens, the T cell co-receptor CD4 and the HIV-1 envelope (Sasso et al., 1989; Shorki et al., 1991; Silvermann et al., 1995; Cleary et al., 1994; Lenert et al., 1990; Berberian et al., 1993; Kreier et al., 1991). In addition, the variable domain is involved in antibody self-binding (Kang et al., 1988), and contains epitopes (idiotopes) recognized by anti-antibodies (Kohler et al., 1989).

Certain examples of antibody conjugates are those conjugates in which the antibody is linked to a detectable label. “Detectable labels” are compounds and/or elements that can be detected due to their specific functional properties, and/or chemical characteristics, the use of which allows the antibody to which they are attached to be detected, and/or further quantified if desired. Another such example is the formation of a conjugate comprising an antibody linked to a cytotoxic or anti cellular agent, and may be termed “immunotoxins”.

Antibody conjugates are used as diagnostic agents. Antibody diagnostics generally fall within two classes, those for use in in vitro diagnostics, such as in a variety of immunoassays, and/or those for use in vivo diagnostic protocols, generally known as “antibody directed imaging”.

Many appropriate imaging agents are known in the art, as are methods for their attachment to antibodies (see, for e.g., U.S. Pat. Nos. 5,021,236; 4,938,948; and 4,472,509, each incorporated herein by reference). The imaging moieties used can be paramagnetic ions; radioactive isotopes; fluorochromes; NMR-detectable substances; X-ray imaging.

In the case of paramagnetic ions, one might mention by way of example ions such as chromium (III), manganese (II), iron (III), iron (II), cobalt (II), nickel (II), copper (II), neodymium (III), samarium (III), ytterbium (III), gadolinium (III), vanadium (II), terbium (III), dysprosium (III), holmium (III) and/or erbium (III). Ions useful in other contexts, such as X-ray imaging, include but are not limited to lanthanum (III), gold (III), lead (II), and especially bismuth (III).

In the case of radioactive isotopes for therapeutic and/or diagnostic application, one might mention astatine211, 14carbon, 51chromium, 36chlorine, 57cobalt, 58cobalt, copper67, 152Eu, gallium67, 3hydrogen, iodine123, iodine125, iodine131, indium111, 59iron, 32phosphorus, rhenium186, rhenium188, 75selenium, 35sulphur, technicium99m and/or yttrium90. 125I is often being commonly used in certain embodiments, and technicium99m and/or indium111 are also often used due to their low energy and suitability for long range detection. Radioactively labeled monoclonal antibodies of the present invention may be produced according to well-known methods in the art. For instance, monoclonal antibodies can be iodinated by contact with sodium and/or potassium iodide and a chemical oxidizing agent such as sodium hypochlorite, or an enzymatic oxidizing agent, such as lactoperoxidase. Monoclonal antibodies according to the invention may be labeled with technetium99m by ligand exchange process, for example, by reducing pertechnate with stannous solution, chelating the reduced technetium onto a Sephadex column and applying the antibody to this column. Alternatively, direct labeling techniques may be used, e.g., by incubating pertechnate, a reducing agent such as SNCl2, a buffer solution such as sodium-potassium phthalate solution, and the antibody. Intermediary functional groups which are often used to bind radioisotopes which exist as metallic ions to antibody are diethylenetriaminepentaacetic acid (DTPA) or ethylene diaminetetracetic acid (EDTA).

Among the fluorescent labels contemplated for use as conjugates include Alexa 350, Alexa 430, AMCA, BODIPY 630/650, BODIPY 650/665, BODIPY-FL, BODIPY-R6G, BODIPY-TMR, BODIPY-TRX, Cascade Blue, Cy3, Cy5,6-FAM, Fluorescein Isothiocyanate, HEX, 6-JOE, Oregon Green 488, Oregon Green 500, Oregon Green 514, Pacific Blue, REG, Rhodamine Green, Rhodamine Red, Renographin, ROX, TAMRA, TET, Tetramethylrhodamine, and/or Texas Red.

Another type of antibody conjugates contemplated in the present invention are those intended primarily for use in vitro, where the antibody is linked to a secondary binding ligand and/or to an enzyme (an enzyme tag) that will generate a colored product upon contact with a chromogenic substrate. Examples of suitable enzymes include urease, alkaline phosphatase, (horseradish) hydrogen peroxidase or glucose oxidase. Secondary binding ligands are biotin and/or avidin and streptavidin compounds. The use of such labels is well known to those of skill in the art and are described, for example, in U.S. Pat. Nos. 3,817,837; 3,850,752; 3,939,350; 3,996,345; 4,277,437; 4,275,149 and 4,366,241; each incorporated herein by reference.

Yet another known method of site-specific attachment of molecules to antibodies comprises the reaction of antibodies with hapten-based affinity labels. Essentially, hapten-based affinity labels react with amino acids in the antigen binding site, thereby destroying this site and blocking specific antigen reaction. However, this may not be advantageous since it results in loss of antigen binding by the antibody conjugate.

Molecules containing azido groups may also be used to form covalent bonds to proteins through reactive nitrene intermediates that are generated by low intensity ultraviolet light (Potter & Haley, 1983). In particular, 2- and 8-azido analogues of purine nucleotides have been used as site-directed photoprobes to identify nucleotide binding proteins in crude cell extracts (Owens & Haley, 1987; Atherton et al., 1985). The 2- and 8-azido nucleotides have also been used to map nucleotide binding domains of purified proteins (Khatoon et al., 1989; King et al., 1989; and Dholakia et al., 1989) and may be used as antibody binding agents.

Several methods are known in the art for the attachment or conjugation of an antibody to its conjugate moiety. Some attachment methods involve the use of a metal chelate complex employing, for example, an organic chelating agent such a diethylenetriaminepentaacetic acid anhydride (DTPA); ethylenetriaminetetraacetic acid; N-chloro-p-toluenesulfonamide; and/or tetrachloro-3-6-diphenylglycouril-3 attached to the antibody (U.S. Pat. Nos. 4,472,509 and 4,938,948, each incorporated herein by reference). Monoclonal antibodies may also be reacted with an enzyme in the presence of a coupling agent such as glutaraldehyde or periodate. Conjugates with fluorescein markers are prepared in the presence of these coupling agents or by reaction with an isothiocyanate. In U.S. Pat. No. 4,938,948, imaging of breast tumors is achieved using monoclonal antibodies and the detectable imaging moieties are bound to the antibody using linkers such as methyl-p-hydroxybenzimidate or N-succinimidyl-3-(4-hydroxyphenyl)propionate.

In other embodiments, derivatization of immunoglobulins by selectively introducing sulfhydryl groups in the Fc region of an immunoglobulin, using reaction conditions that do not alter the antibody combining site are contemplated. Antibody conjugates produced according to this methodology are disclosed to exhibit improved longevity, specificity and sensitivity (U.S. Pat. No. 5,196,066, incorporated herein by reference). Site-specific attachment of effector or reporter molecules, wherein the reporter or effector molecule is conjugated to a carbohydrate residue in the Fc region have also been disclosed in the literature (O'Shannessy et al., 1987). This approach has been reported to produce diagnostically and therapeutically promising antibodies which are currently in clinical evaluation.

B. Immunodetection Methods

In still further embodiments, the present invention concerns immunodetection methods for binding, purifying, removing, quantifying and/or otherwise generally detecting biological components such as a tumor antigen. Some immunodetection methods include enzyme linked immunosorbent assay (ELISA), radioimmunoassay (RIA), immunoradiometric assay, fluoroimmunoassay, chemiluminescent assay, bioluminescent assay, and Western blot to mention a few. The steps of various useful immunodetection methods have been described in the scientific literature, such as, e.g., Doolittle and Ben-Zeev, 1999; Gulbis and Galand, 1993; De Jager et al., 1993; and Nakamura et al., 1987, each incorporated herein by reference.

In general, the immunobinding methods include obtaining a sample suspected of containing tumor antigen, and contacting the sample with a first anti-antibody that reacts immunologically with the antigen in accordance with the present invention, as the case may be, under conditions effective to allow the formation of immunocomplexes.

These methods include methods for purifying an antigen from organelle, cell, tissue or organism's samples. The antibody will preferably be linked to a solid support, such as in the form of a column matrix, and the sample suspected of containing the antibodies that react immunologically with antigen component will be applied to the immobilized antibody. The unwanted components will be washed from the column, leaving the antigen immunocomplexed to the immobilized antibody to be eluted.

The immunobinding methods also include methods for detecting and quantifying the amount of an antigen component in a sample and the detection and quantification of any immune complexes formed during the binding process. Here, one would obtain a sample suspected of containing an antigen, and contact the sample with an antibody that reacts immunologically with the antigen, and then detect and quantify the amount of immune complexes formed under the specific conditions.

In terms of antigen detection, the biological sample analyzed may be any sample that is suspected of containing an antigen, such as, for example, a tissue section or specimen, a homogenized tissue extract, a cell, an organelle, separated and/or purified forms of any of the above antigen-containing compositions, or even any biological fluid that comes into contact with the cell or tissue, including blood and/or serum, although tissue samples or extracts may be used.

Contacting the chosen biological sample with the antibody under effective conditions and for a period of time sufficient to allow the formation of immune complexes (primary immune complexes) is generally a matter of simply adding the antibody composition to the sample and incubating the mixture for a period of time long enough for the antibodies to form immune complexes with, i.e., to bind to antigens present. After this time, the sample-antibody composition, such as a tissue section, ELISA plate, dot blot or western blot, will generally be washed to remove any non-specifically bound antibody species, allowing only those antibodies specifically bound within the primary immune complexes to be detected.

In general, the detection of immunocomplex formation is well known in the art and may be achieved through the application of numerous approaches. These methods are generally based upon the detection of a label or marker, such as any of those radioactive, fluorescent, biological and enzymatic tags. U.S. patents concerning the use of such labels include U.S. Pat. Nos. 3,817,837; 3,850,752; 3,939,350; 3,996,345; 4,277,437; 4,275,149 and 4,366,241, each incorporated herein by reference. Of course, one may find additional advantages through the use of a secondary binding ligand such as a second antibody and/or a biotin/avidin ligand binding arrangement, as is known in the art.

The antibody employed in the detection may itself be linked to a detectable label, wherein one would then simply detect this label, thereby allowing the amount of the primary immune complexes in the composition to be determined. Alternatively, the first antibody that becomes bound within the primary immune complexes may be detected by means of a second binding ligand that has binding affinity for the antibody. In these cases, the second binding ligand may be linked to a detectable label. The second binding ligand is itself often an antibody, which may thus be termed a “secondary” antibody. The primary immune complexes are contacted with the labeled, secondary binding ligand, or antibody, under effective conditions and for a period of time sufficient to allow the formation of secondary immune complexes. The secondary immune complexes are then generally washed to remove any non-specifically bound labeled secondary antibodies or ligands, and the remaining label in the secondary immune complexes is then detected.

Further methods include the detection of primary immune complexes by a two step approach. A second binding ligand, such as an antibody, that has binding affinity for the antibody is used to form secondary immune complexes, as described above. After washing, the secondary immune complexes are contacted with a third binding ligand or antibody that has binding affinity for the second antibody, again under effective conditions and for a period of time sufficient to allow the formation of immune complexes (tertiary immune complexes). The third ligand or antibody is linked to a detectable label, allowing detection of the tertiary immune complexes thus formed. This system may provide for signal amplification if this is desired.

One method of immunodetection designed by Charles Cantor uses two different antibodies. A first step biotinylated, monoclonal or polyclonal antibody is used to detect the target antigen(s), and a second step antibody is then used to detect the biotin attached to the complexed biotin. In that method the sample to be tested is first incubated in a solution containing the first step antibody. If the target antigen is present, some of the antibody binds to the antigen to form a biotinylated antibody/antigen complex. The antibody/antigen complex is then amplified by incubation in successive solutions of streptavidin (or avidin), biotinylated DNA, and/or complementary biotinylated DNA, with each step adding additional biotin sites to the antibody/antigen complex. The amplification steps are repeated until a suitable level of amplification is achieved, at which point the sample is incubated in a solution containing the second step antibody against biotin. This second step antibody is labeled, as for example with an enzyme that can be used to detect the presence of the antibody/antigen complex by histoenzymology using a chromogen substrate. With suitable amplification, a conjugate can be produced which is macroscopically visible.

Another known method of immunodetection takes advantage of the immuno-PCR (Polymerase Chain Reaction) methodology. The PCR method is similar to the Cantor method up to the incubation with biotinylated DNA, however, instead of using multiple rounds of streptavidin and biotinylated DNA incubation, the DNA/biotin/streptavidin/antibody complex is washed out with a low pH or high salt buffer that releases the antibody. The resulting wash solution is then used to carry out a PCR reaction with suitable primers with appropriate controls. At least in theory, the enormous amplification capability and specificity of PCR can be utilized to detect a single antigen molecule.

The immunodetection methods of the present invention have evident utility in the diagnosis and prognosis of conditions such as cancer wherein a specific tumor antigen is expressed. Here, a biological and/or clinical sample suspected of containing a specific disease associated antibody is used. However, these embodiments also have applications to non-clinical samples, such as in the titering of antigen in samples.

In the clinical diagnosis and/or monitoring of patients with various forms a disease, such as, for example, cancer, the detection of a cancer specific antigens, and/or an alteration in the levels of antigens in comparison to the levels in a corresponding biological sample from a normal subject is indicative of a patient with cancer. However, as is known to those of skill in the art, such a clinical diagnosis would not necessarily be made on the basis of this method in isolation. Those of skill in the art are very familiar with differentiating between significant differences in types and/or amounts of biomarkers, which represent a positive identification, and/or low level and/or background changes of biomarkers. Indeed, background expression levels are often used to form a “cut-off” above which increased detection will be scored as significant and/or positive. Of course, the antibodies of the present invention in any immunodetection or therapy known to one of ordinary skill in the art.

1. ELISAs

As detailed above, immunoassays, in their most simple and/or direct sense, are binding assays. Certain immunoassays are the various types of enzyme linked immunosorbent assays (ELISAs) and/or radioimmunoassays (RIA) known in the art. Immunohistochemical detection using tissue sections is also particularly useful. However, it will be readily appreciated that detection is not limited to such techniques, and/or western blotting, dot blotting, FACS analyses, and/or the like may also be used.

In some aspects of the invention, there are ELISA/trastuzumab assays, including in kits, to test samples of subjects that are starting treatment with trastuzumab, to predict response. This may be considered is a new use for a known Ab. In addition, there may be an ELISA/therapeutic Abs kit, to test all at once. In particular, exemplary mAbs that concern the invention include trastuzuman (Herceptin®), cetuximab, (C225 or Erbitux®), rituximab (Rituxan® or Mabthera), Bevacizumab (Avastin®), Edrecolomab (Panorex®), and Alemtuzumab (Campath®).

In one exemplary ELISA, the anti-ORF message and/or anti-ORF translated product antibodies of the invention are immobilized onto a selected surface exhibiting protein affinity, such as a well in a polystyrene microtiter plate. Then, a test composition suspected of containing the antigen, such as a clinical sample, is added to the wells. After binding and/or washing to remove non-specifically bound immune complexes, the bound antigen may be detected. Detection is generally achieved by the addition of another anti-ORF message and/or anti-ORF translated product antibody that is linked to a detectable label. This type of ELISA is a simple “sandwich ELISA”. Detection may also be achieved by the addition of a second anti-ORF message and/or anti-ORF translated product antibody, followed by the addition of a third antibody that has binding affinity for the second antibody, with the third antibody being linked to a detectable label.

In another exemplary ELISA, the samples suspected of containing the antigen are immobilized onto the well surface and/or then contacted with the anti-ORF message and/or anti-ORF translated product antibodies of the invention. After binding and/or washing to remove non-specifically bound immune complexes, the bound anti-ORF message and/or anti-ORF translated product antibodies are detected. Where the initial anti-ORF message and/or anti-ORF translated product antibodies are linked to a detectable label, the immune complexes may be detected directly. Again, the immune complexes may be detected using a second antibody that has binding affinity for the first anti-ORF message and/or anti-ORF translated product antibody, with the second antibody being linked to a detectable label.

Another ELISA in which the antigens are immobilized, involves the use of antibody competition in the detection. In this ELISA, labeled antibodies against an antigen are added to the wells, allowed to bind, and/or detected by means of their label. The amount of an antigen in an unknown sample is then determined by mixing the sample with the labeled antibodies against the antigen during incubation with coated wells. The presence of an antigen in the sample acts to reduce the amount of antibody against the antigen available for binding to the well and thus reduces the ultimate signal. This is also appropriate for detecting antibodies against an antigen in an unknown sample, where the unlabeled antibodies bind to the antigen-coated wells and also reduces the amount of antigen available to bind the labeled antibodies.

Irrespective of the format employed, ELISAs have certain features in common, such as coating, incubating and binding, washing to remove non-specifically bound species, and detecting the bound immune complexes. These are described below.

In coating a plate with either antigen or antibody, one will generally incubate the wells of the plate with a solution of the antigen or antibody, either overnight or for a specified period of hours. The wells of the plate will then be washed to remove incompletely adsorbed material. Any remaining available surfaces of the wells are then “coated” with a nonspecific protein that is antigenically neutral with regard to the test antisera. These include bovine serum albumin (BSA), casein or solutions of milk powder. The coating allows for blocking of nonspecific adsorption sites on the immobilizing surface and thus reduces the background caused by nonspecific binding of antisera onto the surface.

In ELISAs, it is probably more customary to use a secondary or tertiary detection means rather than a direct procedure. Thus, after binding of a protein or antibody to the well, coating with a non-reactive material to reduce background, and washing to remove unbound material, the immobilizing surface is contacted with the biological sample to be tested under conditions effective to allow immune complex (antigen/antibody) formation. Detection of the immune complex then requires a labeled secondary binding ligand or antibody, and a secondary binding ligand or antibody in conjunction with a labeled tertiary antibody or a third binding ligand.

“Under conditions effective to allow immune complex (antigen/antibody) formation” means that the conditions preferably include diluting the antigens and/or antibodies with solutions such as BSA, bovine gamma globulin (BGG) or phosphate buffered saline (PBS)/Tween. These added agents also tend to assist in the reduction of nonspecific background.

The “suitable” conditions also mean that the incubation is at a temperature or for a period of time sufficient to allow effective binding. Incubation steps are typically from about 1 to 2 to 4 hours or so, at temperatures preferably on the order of 25° C. to 27° C., or may be overnight at about 4° C. or so.

Following all incubation steps in an ELISA, the contacted surface is washed so as to remove non-complexed material. A particular washing procedure includes washing with a solution such as PBS/Tween, or borate buffer. Following the formation of specific immune complexes between the test sample and the originally bound material, and subsequent washing, the occurrence of even minute amounts of immune complexes may be determined.

To provide a detecting means, the second or third antibody will have an associated label to allow detection. Preferably, this will be an enzyme that will generate color development upon incubating with an appropriate chromogenic substrate. Thus, for example, one will desire to contact or incubate the first and second immune complex with a urease, glucose oxidase, alkaline phosphatase or hydrogen peroxidase-conjugated antibody for a period of time and under conditions that favor the development of further immune complex formation (e.g., incubation for 2 hours at room temperature in a PBS-containing solution such as PBS-Tween).

After incubation with the labeled antibody, and subsequent to washing to remove unbound material, the amount of label is quantified, e.g., by incubation with a chromogenic substrate such as urea, or bromocresol purple, or 2,2′-azino-di-(3-ethyl-benzthiazoline-6-sulfonic acid (ABTS), or H₂O₂, in the case of peroxidase as the enzyme label. Quantification is then achieved by measuring the degree of color generated, e.g., using a visible spectra spectrophotometer.

2. Immunohistochemistry

The antibodies of the present invention may also be used in conjunction with both fresh-frozen and/or formalin-fixed, paraffin-embedded tissue blocks prepared for study by immunohistochemistry (IHC). The method of preparing tissue blocks from these particulate specimens has been successfully used in previous IHC studies of various prognostic factors, and/or is well known to those of skill in the art (Brown et al., 1990; Abbondanzo et al., 1990; Allred et al., 1990).

Briefly, frozen-sections may be prepared by rehydrating 50 ng of frozen “pulverized” tissue at room temperature in phosphate buffered saline (PBS) in small plastic capsules; pelleting the particles by centrifugation; resuspending them in a viscous embedding medium (OCT); inverting the capsule and/or pelleting again by centrifugation; snap-freezing in 70° C. isopentane; cutting the plastic capsule and/or removing the frozen cylinder of tissue; securing the tissue cylinder on a cryostat microtome chuck; and/or cutting 25-50 serial sections.

Permanent-sections may be prepared by a similar method involving rehydration of the 50 mg sample in a plastic microfuge tube; pelleting; resuspending in 10% formalin for 4 hours fixation; washing/pelleting; resuspending in warm 2.5% agar; pelleting; cooling in ice water to harden the agar; removing the tissue/agar block from the tube; infiltrating and/or embedding the block in paraffin; and/or cutting up to 50 serial permanent sections.

### VI. PHARMACEUTICAL FORMULATIONS AND DELIVERY

The pharmaceutical or antibody compositions disclosed herein may be administered parenterally, intravenously, intradermally, intramuscularly, transdermally or even intraperitoneally as described in U.S. Pat. No. 5,543,158; U.S. Pat. No. 5,641,515 and U.S. Pat. No. 5,399,363 (each specifically incorporated herein by reference in its entirety).

Solutions of the active compounds as free base or pharmacologically acceptable salts may be prepared in water suitably mixed with a surfactant, such as hydroxypropylcellulose. Dispersions may also be prepared in glycerol, liquid polyethylene glycols, and mixtures thereof and in oils. Under ordinary conditions of storage and use, these preparations contain a preservative to prevent the growth of microorganisms. The pharmaceutical forms suitable for injectable use include sterile aqueous solutions or dispersions and sterile powders for the extemporaneous preparation of sterile injectable solutions or dispersions (U.S. Pat. No. 5,466,468, specifically incorporated herein by reference in its entirety). In all cases the form must be sterile and must be fluid to the extent that easy syringability exists. It must be stable under the conditions of manufacture and storage and must be preserved against the contaminating action of microorganisms, such as bacteria and fungi. The carrier can be a solvent or dispersion medium containing, for example, water, ethanol, polyol (e.g., glycerol, propylene glycol, and liquid polyethylene glycol, and the like), suitable mixtures thereof, and/or vegetable oils. Proper fluidity may be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous, intratumoral and intraperitoneal administration. In this connection, sterile aqueous media that can be employed will be known to those of skill in the art in light of the present disclosure. For example, one dosage may be dissolved in 1 ml of isotonic NaCl solution and either added to 1000 ml of hypodermoclysis fluid or injected at the proposed site of infusion, (see for example, “Remington's Pharmaceutical Sciences” 15th Edition, pages 1035-1038 and 1570-1580). Some variation in dosage will necessarily occur depending on the condition of the subject being treated. The person responsible for administration will, in any event, determine the appropriate dose for the individual subject. Moreover, for human administration, preparations should meet sterility, pyrogenicity, general safety and purity standards as required by FDA Office of Biologics standards.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, specific methods of preparation are vacuum-drying and freeze-drying techniques which yield a powder of the active ingredient plus any additional desired ingredient from a previously sterile-filtered solution thereof.

The compositions disclosed herein may be formulated in a neutral or salt form. Pharmaceutically-acceptable salts, include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like. Upon formulation, solutions will be administered in a manner compatible with the dosage formulation and in such amount as is therapeutically effective. The formulations are easily administered in a variety of dosage forms such as injectable solutions, drug release capsules and the like.

As used herein, “carrier” includes any and all solvents, dispersion media, vehicles, coatings, diluents, antibacterial and antifungal agents, isotonic and absorption delaying agents, buffers, carrier solutions, suspensions, colloids, and the like. The use of such media and agents for pharmaceutical active substances is well known in the art. Except insofar as any conventional media or agent is incompatible with the active ingredient, its use in the therapeutic compositions is contemplated. Supplementary active ingredients can also be incorporated into the compositions.

### EXAMPLES

The following examples are included to demonstrate particular embodiments of the invention. It should be appreciated by those of skill in the art that the techniques disclosed in the examples which follow represent techniques discovered by the inventors to function well in the practice of the invention, and thus can be considered to constitute particular modes for its practice. However, those of skill in the art should, in light of the present disclosure, appreciate that many changes can be made in the specific embodiments which are disclosed and still obtain a like or similar result without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### Example 1

### Effects of Allelic Variation on Antibody-Dependent Cell-Mediated Cytotoxicity

Aggregated IgG1 proteins used in the studies detailed here expressed two allelic phenotypes—GM 3+,1−,2− and GM 17+,1+,2+—that differ by four aminoacid residues at positions 214, 356, 358, and 431 of the γ1 chain (Lefranc and Lefranc, 1990). Natural killer (NK) effector cells mediating the antibody-dependent cell-mediated cytotoxicity (ADCC) of HER2-expressing SKBR-3 cells and HER1-expressing A431 cells were either homozygous for the V or F allele at position 158 of the FcγRIIIa protein.

### Inhibition of the Binding of SKBR-3/Trastuzumab Complex to NK Cells by GM 17+,1+,2+ and GM 3+,1−,2− Allotypes of IgG1.

As shown in Table 2, at a concentration of 25 μg/ml, aggregated IgG1 expressing GM 3+,1−,2− allotypes blocked virtually all FcγRIIIa-VV present on the NK cells, resulting in almost 100 percent inhibition of trastuzumab-mediated ADCC of SKBR-3 cells (97.4±0.5%). This phenotype had similar inhibitory effect on ADCC when the NK cells were homozygous for the F allele (94.5±4.2%). In contrast, the inhibitory effect of IgG1 expressing the GM 17+,1+,2+ allotypes was significantly higher when the NK cells were homozygous for the V allele than when they were homozygous for the F allele of FcγRIIIa (73.8±12.8% vs. 27.8±2.1%; p=0.02). Thus, among the GM allotype-FcγRIIIa genotypic combinations studied, a combination of aggregated IgG1 expressing GM 3+,1−,2− allotypes and homozygosity for the FcγRIIIa V allele is the most potent, and IgG1 expressing the GM 17+,1+,2+ allotypes and homozygosity for the FcγRIIIa F allele the least potent, in inhibiting the trastuzumab-mediated ADCC of SKBR-3 cells (97.4±0.5% vs. 27.8±2.1%; p=0.0001).

TABLE 2
Inhibition of trastuzumab-mediated ADCC of SKBR-3 cell
by NK cells expressing different FcγRIIIa genotypes
in the presence of allotypically disparate IgG1 proteins

| IgG1 | FcγRIIIa | Inhibition of | |
|---|---|---|---|
| Phenotype | Genotype | ADCC | p value |
| GM 3+, 1−, 2− | VV | 97.4% ± 0.5% | 0.35 |
| | FF | 94.5% ± 4.2% | |
| GM 17+, 1+, 2+ | VV | 73.8% ± 12.8% | 0.02 |
| | FF | 27.8% ± 2.1% | |
| GM 3+, 1−, 2− | VV | 97.4% ± 0.5% | 0.08 |
| GM 17+, 1+, 2+ | VV | 73.8% ± 12.8% | |
| GM 3+, 1−, 2− | FF | 94.5% ± 4.2% | 0.0001 |
| GM 17+, 1+, 2+ | FF | 27.8% ± 2.1% | |

### Inhibition of the Binding of A431/Cetuximab Complex to NK Cells by GM 17+,1+,2+ and GM 3+,1−,2− Allotypes of IgG1

To determine whether or not the GM allotype-associated differential inhibition of trastuzumab-mediated ADCC of SKBR-3 cells is specific to the cell line and the monoclonal antibody used, these studies were repeated with cetuximab and HER1-over-expressing A431 cells. The results were comparable (Table 3). As with trastuzumab, IgG1 expressing the GM 3+,1−,2− allotypes was equally (albeit not as strongly) effective in inhibiting cetuximab-mediated ADCC of A431 cells, in the presence of NK cells expressing either V or F allele of FcγRIIIa (79.4±5.5% vs. 76.5±2.4%; p=0.47). In contrast—and similar to the results obtained with trastuzumab—IgG1 expressing the allelic GM 17+,1+,2+ allotypes was significantly more effective in inhibiting the cetuximab-mediated ADCC of A431 cells when NK cells expressed the V, rather than the F, allele of FcγRIIIa (83.3±2.6% vs. 50.3±2.9%; p=0.0001). For both therapeutic antibodies, IgG1 expressing the GM 17+,1+,2+ allotypes and homozygosity for the FcγRIIIa F allele was the least potent combination for inhibiting monoclonal antibody-mediated ADCC of target cancer cells.

TABLE 3
Inhibition of cetuximab-mediated ADCC of A431 cell by
NK cells expressing different FcγRIIIa genotypes in
the presence of allotypically disparate IgG1 proteins

| IgG1 | FcγRIIIa | Inhibition of | |
|---|---|---|---|
| Phenotype | Genotype | ADCC | p value |
| GM 3+, 1−, 2− | VV | 79.4% ± 5.5% | 0.47 |
| | FF | 76.5% ± 2.4% | |
| GM 17+, 1+, 2+ | VV | 83.3% ± 2.6% | 0.0001 |
| | FF | 50.3% ± 2.9% | |
| GM 3+, 1−, 2− | VV | 79.4% ± 5.5% | 0.35 |
| GM 17+, 1+, 2+ | VV | 83.3% ± 2.6% | |
| GM 3+, 1−, 2− | FF | 76.5% ± 2.4% | 0.0002 |
| GM 17+, 1+, 2+ | FF | 50.3% ± 2.9% | |

### Discussion

Association of certain Fc (GM)-FcγRIIIa genotypic combinations with higher inhibition of NK-mediated ADCC observed here might be a reflection of higher affinity between Fc and FcγRIIIa molecules expressing these genotypes. In the presence of both FcγRIIIa-VV and FcγRIIIa-FF expressing NK cells, the inhibitory effect on trastuzumab-mediated ADCC was higher for the IgG1 expressing GM 3+,1−,2− allotypes than that for IgG1 expressing the GM 17+,1+,2+ allotypes. This appears to be consistent with the results of a recent investigation on the measurement of IgG-FcγRIIIa binding (Armour et al., 2010). These authors reported that for both FcγRIIIa V and F expressing cells, IgG proteins of GM 3 allotype bound slightly but reproducibly better than those expressing the GM 1,17 allotypes.

Studies presented here have important implications for antibody-based therapy in cancer patients. Musolino et al. (2008) have reported that homozygosity for the V allele of FcγRIIIa, which was significantly correlated with objective response rate and progression free survival, was also associated with significantly higher level of trastuzumab-mediated ADCC. The studies here are consistent with this finding, as the constant region of trastuzumab is similar to the GM 17+,1+,2+ phenotype in that it carries the GM 17 allotype, although it lacks the GM 1 and 2 determinants. (The GM 1 allotype, which is in almost absolute linkage disequilibrium with GM 17 in Caucasians, was engineered out and replaced with the non-GM 1 isoallotype, to make trastuzumab less immunogenic (Klein, 2009). The results reported by Musolino et al. (2008) imply that majority of the people, who carry the more common F allele (Lehrnbecher et al., 1999), are unlikely to respond adequately to this therapy. The studies presented here found that IgG1 proteins expressing GM 3+,1−,2− allotypes had high inhibitory effect on ADCC in the presence of both VV and FF expressing NK cells. This suggests that a HER2-targetting humanized monoclonal antibody, which expresses the GM 3+,1−,2− allotypes in the constant region, would be beneficial to the entire patient population, and especially to those (F-carriers) who are unlikely to respond to the current trastuzumab-based therapy.

Monoclonal therapeutic antibodies with engineered/mutated Fc variants have shown dramatically higher ADCC in vitro (Lazar et al., 2006); however, as mentioned above, any harmful in vivo consequences of increased immunogenicity of these antibodies have not been adequately investigated. Therapeutic IgG antibodies with naturally occurring Fc variants (GM allotypes) are less likely to be immunogenic than those carrying the engineered variants. Therefore, to obtain the maximum clinical efficacy of humanized monoclonal antibodies, further studies are needed to explore the role of GM and FcγR loci in ADCC. Genes do not act in isolation: there is growing body of evidence that epistasis—modification of the action of a gene by one or more other genes—plays a significant role in many structural and regulatory pathways (Phillips, 2008). Determinants expressed on Fc and FcγR are probably some of the most likely ligand-receptor candidate pairs for gene-gene interactions in the human genome, and based on other allelic ligand-receptor interactions involving genes of the immune system (Khakoo et al., 2004), it is reasonable to expect that such interactions contribute to the immunological pathways leading to the ADCC of tumor cells.

### Example 2

### Materials and Methods

### Allotyping, Affinity Purification, and Heat Aggregation of IgG1 Proteins

Serum samples from healthy blood donors were allotyped for all four known IgG1 allotypes—GM 1/a, 2/x, 3/f, and 17/z—by a standard hemagglutination-inhibition assay (Schanfield and van Loghem, 1986). The study protocol was approved by the local Institutional Review Board for human research. Total IgG from the pooled sera of subjects—10 expressing the GM 3+,1−,2− and 10 expressing the alternative GM 17+,1+,2+ allotypes—was concentrated by ammonium sulfate fractionation. Allotypes 3 and 17 are expressed in the Fd, whereas 1 and 2 are expressed in the Fc, region of the γ chains. These contrasting allelic combinations provide the maximum possible allotypic differences between the two IgG1 preparations to be used in the ADCC inhibition assays. The GM allotype notation follows the international system for human gene nomenclature (Shows et al., 1987), incorporated herein by reference.

IgG1 proteins were isolated from the total IgG by subclass-specific affinity chromatography, and purity of the preparation was tested by human IgG subclass specific ELISA. Interactions between monomeric IgG and low-affinity FcγRs are unstable and require multivalent interactions for their persistence (Maenaka et al., 2001). To make the interaction between the low-affinity FcγRIIIa and IgG1 more stable, the affinity purified IgG1 was heat aggregated, following the protocol of others who have used ADCC inhibition assays (MacDonald et al., 1981) or have investigated IgG-FcγR interactions (Tamm et al., 1996).

### FcγRIIIa Genotyping

A change in the nucleotide at position 559 of the FcγRIIIa gene from T to G results in amino acid change from phenylalanine (F) to valine (V) at position 158 in the IgG binding domain of FcγRIIIa. This biallelic functional polymorphism alters the level of interaction between the receptor and its IgG ligands, resulting in the modulation of the IgG-mediated effector functions (Koene et al., 1997; Carton et al., 2002). Genotyping of FcγRIIIa alleles (dbSNP: rs396991) was performed by real time polymerase chain reaction (RT-PCR), using a pre-designed TagMan® genotyping assay from Applied Biosystems Inc. (Foster City, Calif.). RT-PCR assays were performed in triplicate, using a Bio-Rad iCycler (Bio-Rad Laboratories, Hercules, Calif.) and following the annealing and extension conditions provided in the TaqMan 0 assay protocol.

### Purification of NK Cells

Blood from volunteers homozygous for either V or F alleles of FcγRIIIa was collected in EDTA-coated vacutainer tubes (BD, Franklin Lakes, N.J.). White blood cells were isolated by density gradient centrifugation using Histopaque-1077 (Sigma-Aldrich Corp., St. Louis, Mo.). Cells were washed and cultured overnight in RPMI containing 10% fetal bovine serum (FBS) and 10% Human AB serum. NK cells were isolated by affinity depletion of non-NK cells using a kit from Milteneyi Biotec (Auburn, Calif.), according to the manufacturer's protocol.

### Cell Cytotoxicity by ADCC and ADCC Inhibition

This method is analogous, in principle, to that described by Macdonald et al. (1981), incorporated herein by reference. Human breast cancer cell line SKBR-3, which over-expresses HER2, and epidermal cancer cell line A431, which over expresses EGFR/HER1, were obtained from ATCC (Manassas, Va.). Cells were cultured overnight in medium (McCoy's 5A and DMEM for SKBR-3 and A431, respectively) containing 10% FBS, harvested with trypsin-EDTA, and washed 3 times with RPMI-1640 containing 1% BSA. Cells were coated with 10 μg/ml target binding antibody (trastuzumab for SKBR-3 and cetuximab for A431) for 30 min on ice and washed three times with RPMI 1640 containing 1% BSA. These coated cells (5×10³) were further incubated with 25 μg/ml of aggregated human IgG1—expressing either GM 3+,1−,2− or GM 17+,1+,2+ allotypes—and 5×10⁴ NK cells (1:10 ratio of target to effector cells) in a volume of 100 μl RPMI 1640 containing 1% BSA in 96-well plates in triplicate for 4 hrs. The plates were then centrifuged and the supernatant was assayed for lactate dehydrogenase (LDH) activity using the Cytotox-96 kit from Promega Corporation (Madison, Wis.). Spontaneous LDH release—possibly due to killer-cell immunoglobulin-like receptor dependent cytotoxicity—from target cells incubated with NK cells, was used as blank (negative control).

$$A D C C inhibition ( \% ) = \frac{( Control L D H activity - Test L D H activity )}{( Control L D H activity )} \times 100$$

Where the Test involves target cells incubated with aggregated IgG1, target binding antibody, and NK cells and control (positive) consists of target cells incubated with target binding antibody and NK cells.

Standard deviations were obtained via experiment replication. Each experiment was done in triplicate and repeated three times.

### Statistical Analysis

Student's unpaired t-Test was used to compare the percentage of ADCC inhibition associated with GM-FcγRIIIa genotypic combinations. All tests were two-tailed, and the statistical significance was defined as p<0.05.

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
U.S. Pat. No. 3,817,837
U.S. Pat. No. 3,850,752
U.S. Pat. No. 3,939,350
U.S. Pat. No. 3,996,345
U.S. Pat. No. 4,196,265
U.S. Pat. No. 4,275,149
U.S. Pat. No. 4,277,437
U.S. Pat. No. 4,366,241
U.S. Pat. No. 4,472,509
U.S. Pat. No. 4,938,948
U.S. Pat. No. 5,021,236
U.S. Pat. No. 5,196,066
U.S. Pat. No. 5,399,363
U.S. Pat. No. 5,466,468
U.S. Pat. No. 5,543,158
U.S. Pat. No. 5,641,515
U.S. Pat. No. 6,150,584
U.S. Patent Publn. 20110052570
Abbondanzo et al., Breast Cancer Res. Treat., 16:182(151), 1990.
Allred et al., Breast Cancer Res. Treat., 16:182(149), 1990.
Armour et al., J. Immunol. Methods, 354:20-33, 2010.
Atherton et al., Biol. Reprod., 32(1):155-171, 1985.
Berberian et al., Science, 261:1588-1591, 1993.
Bird et al., Science, 242:423-426, 1988.
Bowie et al., Science, 253(5016):164-170, 1991
Brown et al. Immunol. Ser., 53:69-82, 1990.
Campbell, In: Monoclonal Antibody Technology, Laboratory Techniques in Biochemistry and Molecular Biology, Burden and Von Knippenberg (Eds.), Elseview, Amsterdam, 75-83, 1984.
Cartron et al., Blood, 99:754-758, 2002.
Cleary et al., Trends Microbiol., 4:131-136, 1994.
De Jager et al., Semin. Nucl. Med., 23(2):165-179, 1993.
Doolittle and Ben-Zeev, Methods Mol, Biol, 109:215-237, 1999.
Gefter et al., Somatic Cell Genet., 3:231-236, 1977.
Goding, In: Monoclonal Antibodies: Principles and Practice, 2d ed., Academic Press, Orlando, Fla., 71-74, 1986.
Goding, In: Monoclonal Antibodies: Principles and Practice, 2nd ed., Academic Press, Orlando, Fla., pp 65-66, 1986.
Goding, In: Monoclonal Antibodies: Principles and Practice, 2^{nd} Ed., 1986.
Gulbis and Galand, Hum. Pathol., 24(12):1271-1285, 1993.
Harlow and Lane, In: Antibodies: A Laboratory Manual, Cold Spring Harbor Laboratory, Cold Spring Harbor, N.Y., 346-348, 1988.
Jakobovits et al., Nature, 362:255-258, 1993.
Jones et al., Nature, 321:522-525, 1986.
Kabat et al., In: Sequences of Proteins of Immunological Interest, 5^{th} Ed., Public Health Service, National Institute of Health, Bethesda, Md., 1991.
Khakoo et al., Science, 305:872-874, 2004.
Khatoon et al., Ann. Neurol, 26(2):210-215, 1989.
King et al., J. Biol. Chem., 269, 10210-10218, 1989.
King et al., J. Biol. Chem., 269, 10210-10218, 1989.
Klein Proc. Natl. Acad. Sci. USA, 10:859-863, 2009.
Koene et al., Blood, 90:1109-1114, 1997.
Kohler and Milstein, Eur. J. Immunol., 6:511-519, 1976.
Kohler and Milstein, Nature, 256:495-497, 1975.
Kohler et al., Methods Enzymol, 178:3-35, 1989.
Kozbor, J. Immunol., 133(6):3001-3005, 1984.
Kreier et al., In: Infection, Resistance and Immunity, Harper and Row, N.Y., 1991.
Lazar et al., Proc. Natl. Acad. Sci. USA, 103:4005-4010, 2006.
Lefranc and Lefranc, In: Molecular genetics of immunoglobulin allotype expression, Shakib (Ed), The Human IgG Subclasses: Molecular Analysis of Structure, Function and Regulation, Oxford: Pergamon Press, London, pp 3-78, 1990.
Lehrnbecher et al., Blood, 94:4220-4232, 1999.
Lenert et al., Science, 248:1639-1643, 1990.
MacDonald et al., J. Immunol. Methods, 46:69-76, 1981.
Maenaka et al., J. Biol. Chem., 276:44898-44904, 2001.
McCafferty et al., Nature, 348:552-553, 1990.
Musolino et al., J. Clin. Oncol., 26:1789-1796, 2008.
Nakamura et al., In: Handbook of Experimental Immunology (4^{th} Ed.), Weir et al. (Eds), 1:27, Blackwell Scientific Publ., Oxford, 1987.
Nimmerjahn and Ravetch, Nat. Rev. Immunol., 8(1):34-47, 2008.
Nimmerjahn and Ravetch, Nat. Rev. Immunol., 8(1):34-47, 2008. O'Shannessy et al., J. Immun. Meth., 99, 153-161, 1987.
Owens and Haley, J. Biol. Chem., 259:14843-14848, 1987.
Phillips, Nat. Rev. Genet., 9:855-867, 2008.
Potter and Haley, Methods Enzymol, 91:613-633, 1983.
Remington's Pharmaceutical Sciences” 15^{th} Ed., 1035-1038 and 1570-1580, 1990.
Remington's Pharmaceutical Sciences, 18th Ed. Mack Printing Company, 1289-1329, 1990.
Riechmann et al., Nature, 332:323-329, 1988.
Sasso et al., J. Immunol., 142:2778-2783, 1989.
Schanfield and van Loghem, In: Human immunoglobulin allotypes, Weir (Ed.), Handbook of Experimental Immunology, Blackwell, Boston, 94.1-18.16, 1986.
Shorki et al., J. Immunol., 146:936-940, 1991.
Shows et al., Cytogenet. Cell Genet., 46:11-28, 1987.
Silvermann et al., J. Clin. Invest., 96:417-426, 1995.
Tamm et al., J. Biol. Chem., 271:3659-3666, 1996.
Verhoeyen et al., Science, 239:1534-1536, 1988.

What is claimed is:

1. A pharmaceutical composition comprising a purified EGFR-binding antibody which has a human immunoglobulin heavy constant gamma-1 region with arginine at position 214 and a pharmaceutically acceptable carrier.

2. The composition of claim 1, wherein the human immunoglobulin heavy constant gamma-1 region has glutamic acid, methionine, and alanine at positions 356, 358, and 431, respectively.

3. The composition of claim 1, wherein the human immunoglobulin heavy constant gamma-1 region is encoded by a human G1M3 allele.

4. The composition of claim 1, wherein the antibody is a humanized antibody.

5. The composition of claim 1, wherein the antibody is a human antibody.

6. The composition of claim 1, wherein the antibody is a chimeric antibody.

7. The composition of claim 1, wherein the EGFR is HER2 (human epidermal growth factor receptor 2).

8. The composition of claim 1, wherein the EGFR is HER1 (human epidermal growth factor receptor 1).

9. The composition of claim 1, wherein the antibody is coupled to a therapeutic, a reporter, or a targeting moiety.

10. The composition of claim 9, wherein the therapeutic is a nucleotide, a peptide, a small molecule, a therapeutic radionuclide, a chemotherapeutic, a tumor suppressor, an apoptosis inducer, an enzyme, a second antibody, an siRNA, a hormone, a prodrug, or an immunostimulant.

11. A method of treating epidermal growth factor receptor (EGFR)-expressing cancer in a human, comprising administering to the human a therapeutically effective amount of a pharmaceutical composition of claim 1, wherein the cancer in the human expresses an EGFR.

12. The method of claim 11, further comprising determining FcγRIIIA gene allelic pattern in the human.

13. The method of claim 12, wherein the allelic pattern is determined by allele specific hybridization, primer specific extension, oligonucleotides ligation assay, restriction enzyme site analysis, or single-stranded conformation polymorphism analysis.

14. The method of claim 11, wherein the human has an Fc gamma receptor IIIA (FcγRIIIA) gene determined to have two alleles encoding phenylalanine (F) at position 158.

15. The method of claim 11, wherein the human has an FcγRIIIa gene determined to have two alleles encoding valine (V) at position 158.

16. The method of claim 11, wherein the cancer is a cancer of breast, bladder, brain, lung, liver, spleen, kidney, lymph node, small intestine, pancreas, blood cells, colon, stomach, breast, endometrium, prostate, testicle, ovary, skin, head and neck, esophagus, bone marrow or blood.

17. The method of claim 16, wherein the cancer is breast cancer.

18. The method of claim 11, wherein administering to the human comprises intravenous administration, intracardiac administration, intradermal administration, intralesional administration, intrathecal administration, intracranial administration, intrapericardial administration, intraumbilical administration, intraocular administration, intraarterial administration, intraperitoneal administration, intraosseous administration, intrahemmorhage administration, intratrauma administration, intratumor administration, subcutaneous administration, intramuscular administration, intravitreous administration, direct injection into a normal tissue, or direct injection into a tumor.

19. The method of claim 11, wherein the human has been previously determined to comprising at least one allele of the FcγRIIIA gene that encodes a phenylalanine (F) at position 158.

20. The method of claim 19, wherein the human has been previously determined to comprising two alleles of the FcγRIIIA gene that encode a phenylalanine (F) at position 158.
